(19) United States
(12) Patent Application Publication
(10) Pub. No.: US 2016/0242948 A1
(43) Pub. Date: Aug. 25, 2016

(54) BACK PLATE OF WAIST SUPPORT

(71) Applicant: Meng-Chun WANG, Taichung City (TW)

(72) Inventor: Meng-Chun WANG, Taichung City (TW)

(21) Appl. No.: 14/626,213

(22) Filed: Feb. 19, 2015

(51) Int. Cl. A61F 5/02; A61F 5/26

(52) U.S. Cl. CPC A61F 5/028; A61F 5/26; A61F 2005/0197

(57) ABSTRACT

A back plate of a waist support includes a plate body and two spring members. The plate body has a valley portion and two positioning holes. The two positioning holes are arranged symmetrically on the right and left sides of the valley portion. Each of the spring members is installed in the positioning hole of the plate body and provides a propping force to a wearer of the waist support. Thereby, the waist support can more effectively distribute the load that is otherwise focused on of the wearer's waist vertebra and in turn provides the wearer with more comfort.

### BACKGROUND OF THE INVENTION

1. Technical Field

The present invention relates to waist supports, and more particularly to a back plate of a waist support.

2. Description of Related Art

For those having low-back pain, a waist support is an effective medical aid that eases the wearer's discomfort by distributing loads around his/her waist vertebra. Thus, a patient having low-back pain is usually prescribed to wear a waist support in addition to performing rehabilitation and/or receiving treatment, with the expectation that his/her waist is supported and the effects of the rehabilitation/treatment can be enhanced.

A conventional waist support is mainly constructed from two elastic strips that are put around a wearer's waist and a back plate body that is provided between the two elastic strips and supports the wearer's low back. The known back plate is structurally a rigid board with or without some metal ribs therein for providing supplementary support. However, such a structural configuration is not adequate for providing effective support. Furthermore, over long-term use, it may make its wearer uncomfortable and even have adverse effect on the wearer's rehabilitation.

### BRIEF SUMMARY OF THE INVENTION

The primary objective of the present invention is to provide a back plate of a waist support, wherein the back plate effectively distributes the load that is otherwise focused on of the wearer's waist vertebra and in turn provides the wearer with more comfort.

For achieving the foregoing objective, the disclosed back plate comprises a plate body and at least two spring members. The plate body has a valley portion for receiving a wearer's spine. The plate body further has at least two positioning holes that are arranged symmetrically on two sides of the valley portion. Each of the positioning holes is for receiving one of the spring members. Thereby, in use, the back plate of the present invention implements the plate body to effectively support its wearer's low back and have the spring members prop the wearer's low back additionally, so as to effectively distribute the load that is otherwise focused on of the wearer's waist vertebra and in turn provides the wearer with more comfort.

In one embodiment of the present invention, each of the spring members has a propping ring, a seat, and two curved arms. The propping ring serves to prop the wearer's low back, and the seat is deposited in the positioning hole of the plate body. The two curved arms are symmetrically connected between the propping ring and the seat. Thereby, the spring member provides adequate support to the wearer's low back in virtue of the elastic deformation of the two curved arms.

In one embodiment of the present invention, a hole wall defining the positioning hole of the plate body includes an inner flange, and the seat of each said spring member includes a large-diameter ring-like section and a small-diameter shaft-like section, in which the large-diameter ring-like section has a periphery connected with one end of each said curved arm, while the small-diameter shaft-like section has one end connected to the large-diameter ring-like section and an opposite end inserted in the positioning hole of the plate body, and the small-diameter shaft-like section has an outer flange configured for fittingly engaging with the inner flange of the positioning hole of the plate body. Thereby, the spring member can be received in the positioning hole of the plate body securely.

### BRIEF DESCRIPTION OF THE SEVERAL VIEWS OF THE DRAWINGS

FIG. 1 is an applied view of the present invention working with elastic strips.

FIG. 2 is an exploded view of the present invention.

FIG. 3 is a perspective view of a spring member of the present invention.

FIG. 4 is a partial, cross-sectional view of the present invention.

### DETAILED DESCRIPTION OF THE INVENTION

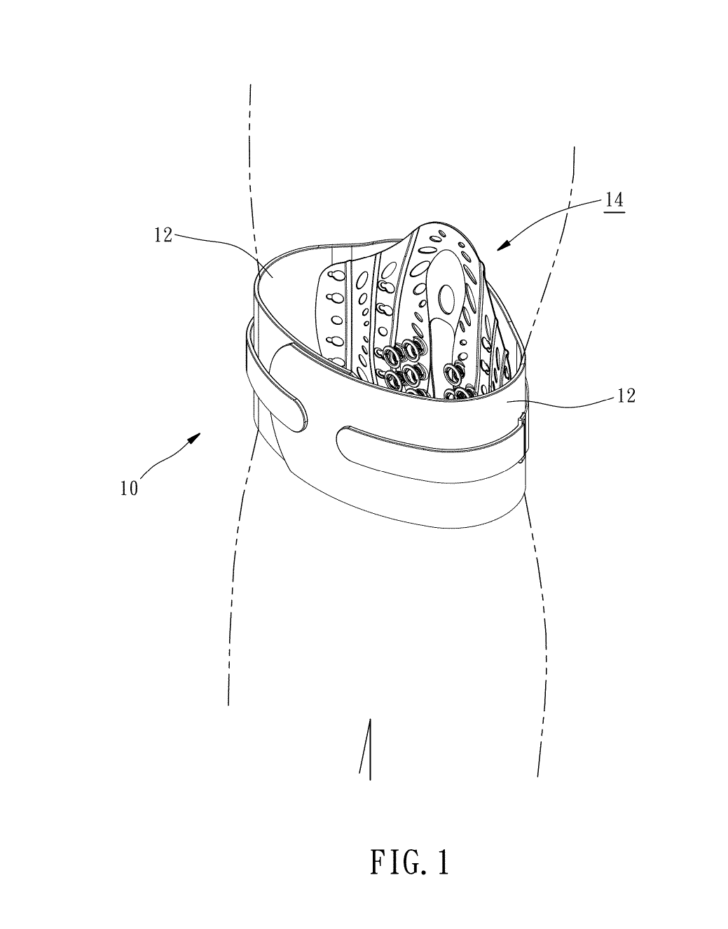

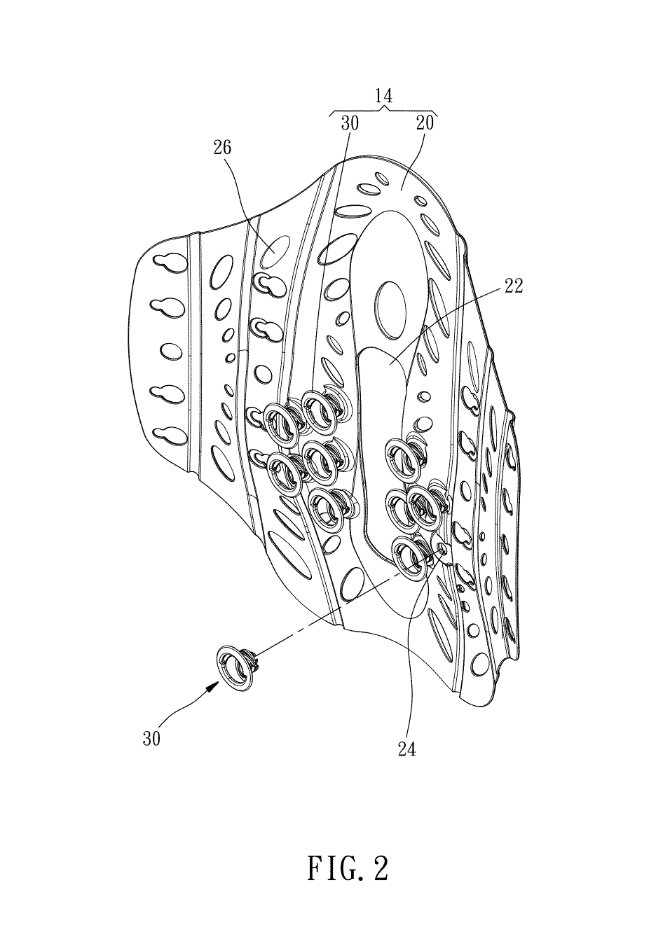

Referring to FIG. 1, a waist support 10 primarily comprises two elastic strips 12 and a back plate 14. As shown in FIG. 2, the disclosed back plate 14 has a plate body 20 and plural spring members 30.

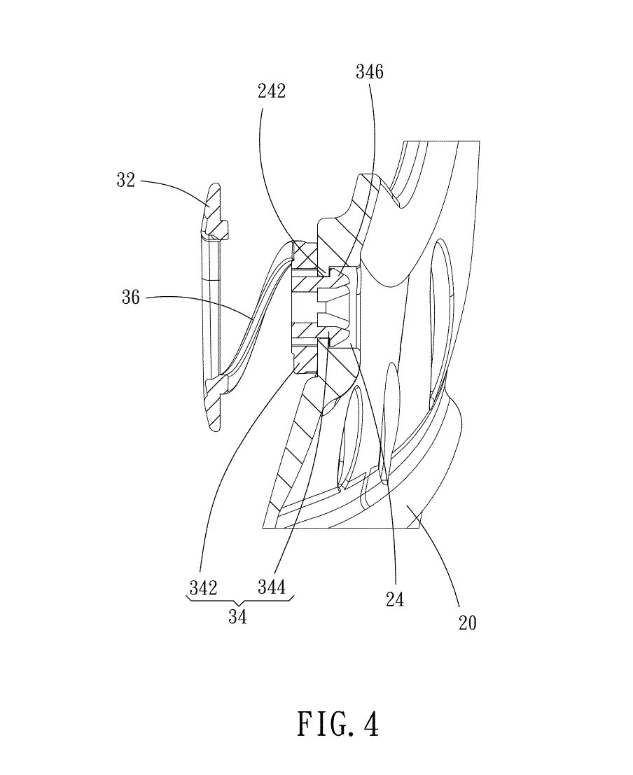

The plate body 20 is arranged between the two elastic strips 12. The plate body 20 is centrally formed with a valley portion 22 for receiving a wearer's spine. The plate body 20 has plural positioning holes 24 that are arranged symmetrically on the right and left sides of the valley portion 22. The positioning holes 24 are spaced along a direction in which the valley portion 22 extends. Additionally, each of the positioning holes 24 is defined by a hole wall on which an inner flange 242 (as shown in FIG. 4) is formed. The plate body 20 has plural hollowed-out portions 26, each for reducing the overall weight of the plate body 20 and allowing ventilation.

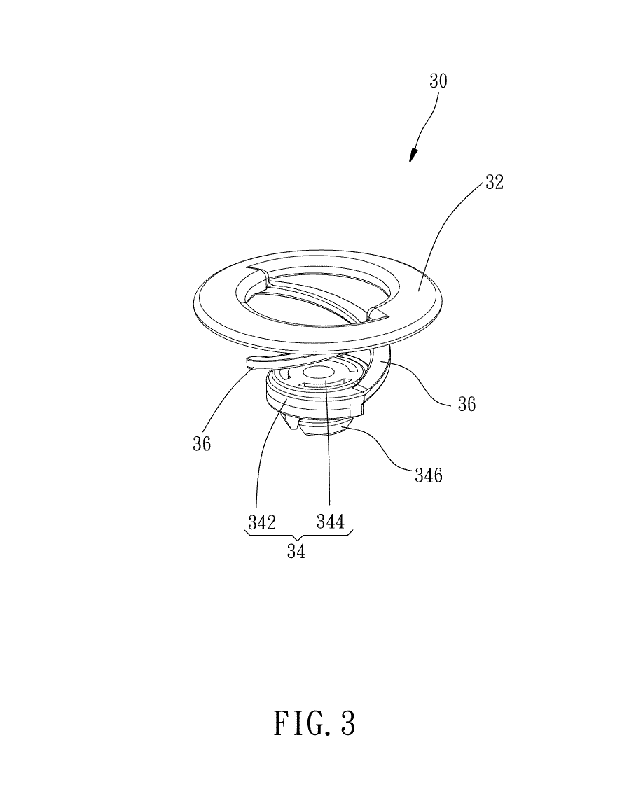

As shown in FIG. 3, each of the spring members 30 has a propping ring 32, a seat 34, and two curved arms 36. The seat 34 includes a large-diameter ring-like section 342 and a small-diameter shaft-like section 344. The small-diameter shaft-like section 344 has one end connected to the large-diameter ring-like section 342. The small-diameter shaft-like section 344 has its opposite end formed with an outer flange 346. The two curved arms 36 are arranged symmetrically. The two curved arms 36 each have one end connected to the inner periphery of the propping ring 32 and an opposite end connected to the outer periphery of the large-diameter ring-like section 342 of the seat 34, thereby pushing the propping ring 32 away from the plate body 20. During fabrication, the spring member 30 has the small-diameter shaft-like section 344 of the seat 34 inserted into the positioning hole 24 of the plate body 20, so that the outer flange 346 of the small-diameter shaft-like section 344 of the seat 34 fittingly engages with the inner flange 242 of the positioning hole 24 of the plate body 20 (as shown in FIG. 4), thereby making the spring member 30 and the plate body 20 combined with each other securely.

When wearing the waist support 10, a wearer first puts the two elastic strips 12 around his/her waist, and then adjust the position of the plate body 20 so as to align his/her spine with the valley portion 22 of the plate body 20. At last, when the two elastic strips 12 are bound with each other, the wearing of the waist support 10 is finished. The properly worn waist support 10 can have the plate body 20 of the back plate 14 support the wearer's low back effectively. Also, with the hollowed-out portions 26 of the plate body 20, ventilation is allowed, so the wearer can wear the waist support 10 for a longer period without feeling uncomfortable. Moreover, since the spring members 30 of the back plate 14 uses the propping rings 32 to better support the wearer's low back, the waist support 10 can more effectively distribute the load that is otherwise focused on of the wearer's waist vertebra and in turn provides the wearer with more comfort.

What is claimed is:

1. A back plate of a waist support, comprising:
a plate body, having a valley portion and at least two positioning holes, wherein the two positioning holes are arranged symmetrically on two sides of the valley portion; and
at least two spring members, each received in one said positioning hole of the plate body.

2. The back plate of claim 1, wherein each of the spring members has a propping ring, a seat, and two curved arms, in which the seat is deposited in the positioning hole of the plate body, and the two curved arms are arranged symmetrically and connected between the propping ring and the seat.

3. The back plate of claim 2, wherein a hole wall defining the positioning hole of the plate body includes an inner flange, and the seat of each said spring member includes a large-diameter ring-like section and a small-diameter shaft-like section, in which the large-diameter ring-like section has a periphery connected with one end of each said curved arm, while the small-diameter shaft-like section has one end connected to the large-diameter ring-like section and an opposite end inserted in the positioning hole of the plate body, and the small-diameter shaft-like section has an outer flange configured for fittingly engaging with the inner flange of the positioning hole of the plate body.

4. The back plate of claim 1, wherein the positioning holes of the plate body are more than two, and the positioning hole are arranged symmetrically on the two sides of the valley portion and spaced along a direction in which the valley portion extends.

5. The back plate of claim 2, wherein the positioning holes of the plate body are more than two, and the positioning hole are arranged symmetrically on the two sides of the valley portion and spaced along a direction in which the valley portion extends.

6. The back plate of claim 3, wherein the positioning holes of the plate body are more than two, and the positioning hole are arranged symmetrically on the two sides of the valley portion and spaced along a direction in which the valley portion extends.
